(19) United States
(12) Patent Application Publication
(10) Pub. No.: US 2024/0366599 A1
(43) Pub. Date: Nov. 7, 2024

(54) HUMAN EFFICACIOUS DOSE AND DOSAGE SCHEDULE OF SPR720

(71) Applicant: SPERO THERAPEUTICS, INC., Cambridge, MA (US)

(72) Inventors: Angela TALLEY, Cambridge, MA (US); David MELNICK, Cambridge, MA (US)

(21) Appl. No.: 18/646,893

(22) Filed: Apr. 26, 2024

Related U.S. Application Data
(63) Continuation of application No. PCT/US2022/047865, filed on Oct. 26, 2022.
(60) Provisional application No. 63/272,052, filed on Oct. 26, 2021.
(60) Provisional application No. 63/300,145, filed on Jan. 17, 2022.

(51) Int. Cl. A61K 31/506; A61K 31/133; A61K 31/438; A61K 31/496; A61K 31/7048; A61K 31/7052; A61P 31/04

(52) U.S. Cl. CPC A61K 31/506; A61K 31/133; A61K 31/438; A61K 31/496; A61K 31/7048; A61K 31/7052; A61P 31/04

(57) ABSTRACT

The present disclose provides methods of treating NTM-PD and other bacterial infections, including pulmonary tuberculous and nontuberculous mycobacteria infections. Clinical studies have established the safety of SPR720 up to daily oral dosages of 2000 mg and shown that once daily oral dosing is sufficient to provide efficacious plasma levels of the active agent, SPR719. Clinical studies have also shown that there is no significant food effect for SPR720 and that the drug may be given in to a patient in the fed or fasted state. Clinical studies have also established that SPR720 can be administered safely to elderly patients. The present disclosure provides a method of treating a bacterial infection in a patient comprising administering a daily oral dose of SPR720, or a pharmaceutically acceptable salt thereof, the oral dose comprising 100 mg SPR720 to 1500 mg SPR720.

### CROSS REFERENCE TO RELATED APPLICATION

This application is a national stage filing of PCT/US2022/047865, which claims priority to U.S. Provisional Appl. No. 63/272,052, filed Oct. 26, 2021, and 63/300,145, filed Jan. 17, 2022, both of which are hereby incorporated by reference in their entirety.

### BACKGROUND

Nontuberculous mycobacteria pulmonary disease (NTM-PD) is a chronic, progressive disease that occurs through inhalation of mycobacteria from environmental sources. Among numerous NTM species worldwide, NTM-PD is primarily caused by Mycobacterium avium complex (MAC) which includes M. avium, M. intracellulare, M. chimaera and several subspecies; M. abscessus and M. kansasii. No systemic oral antimicrobial agents are approved for the treatment of pulmonary nontuberculous mycobacteria infections. Increasing rates of resistance to current standard of care agents, along with tolerability issues, and high rates of clinical relapse, highlight the urgent need for new antimicrobials to treat NTM-PD and also pulmonary tuberculosis.

Nontuberculous mycobacterial pulmonary disease predominantly occurs in patients with underlying lung disease, including chronic obstructive pulmonary disease and bronchiectasis, patients with cystic fibrosis, older males, and nonsmoking women over the age of 50 years without previously diagnosed lung disease (Lady Windermere syndrome). Development of NTM-PD is based on a variety of pathogen and host-specific factors for which the pathophysiology is poorly understood. Risk factors for disease include underlying immunodeficiency, treatment with antitumor necrosis factor agents, and exposure to water sources contaminated with nontuberculous mycobacteria. The natural history of this disease is varied based on pathogen and host factors, but 5-year mortality rates as high as 35% have been reported for M. abscessus pulmonary disease. Risk factors for progressive lung disease include infection with M. abscessus, the presence of cavitary lung disease, and low body weight/body mass index. Based on the patient- and pathogen-specific factors contributing to development of disease, there is significant variability in the clinical presentation of patients with NTM-PD, and definitive diagnostic criteria are lacking. As a result, recommendations in current treatment guidelines for pulmonary NTM infections are based predominantly on observational clinical data supported by in vitro susceptibility data and expert opinion. Understanding the epidemiology and treatment of these infections is complicated by the large number of causative species, the varied clinical manifestations of disease (fibronodular, cavitary, bronchiectatic, etc.), and geographical variation in the prevalence of infection. Patients at the US Food and Drug Administration (FDA) NTM Open Public Meeting (CDER 2016,) described debilitating symptoms such as a relentless and severe deep cough, hemoptysis, progressive fatigue, stabbing chest pain, loss of appetite, along with fever, night sweats, and weight loss, which often indicate advanced disease. In the results of a survey conducted by the NTM Info & Research patient advocacy group and presented at the FDA Workshop, Development of Antibacterial Drugs for Treatment of Nontuberculous Mycobacterial Disease (NTMir 2019), the top symptoms that plagued respondents and had the greatest impact on their lives were fatigue, cough, shortness of breath, night sweats, and weight loss. When asked about their preferences in treatment outcomes, the majority of the respondents selected improving quality of life, increasing energy, and reducing fatigue. Clinical diagnosis of NTM-PD is based on assessment of specific clinical, radiographic and microbiologic criteria as outlined in the 2007 American Thoracic Society and Infectious Disease Society of America treatment guidelines.

Therapy for NTM infections generally requires prolonged treatment (months to years) with antibiotic combination regimens that are poorly tolerated and, in many cases, ineffective. The therapeutic regimens vary based on the specific causative pathogen (MAC, M. abscessus, M. kansasii), the presence or absence of pulmonary cavities at the initiation of therapy, the response to prior attempts at therapy, comorbidities such as renal dysfunction, and the individual patient's ability to tolerate oral, aerosolized, or intravenous (IV) therapy. There is currently only one antimicrobial agent, ARIKAYCE, that is approved for the treatment of NTM-PD. It is approved for patients with MAC pulmonary disease (MAC-PD) as part of a combination antibacterial drug regimen in adult patients who do not achieve negative sputum cultures after a minimum of 6 consecutive months of a multidrug background regimen therapy and who have limited or no alternative treatment options. Current standard of care (SOC) therapy for MAC-PD in the US, as outlined in the joint treatment guidelines developed in 2007 by the American Thoracis Society and Infectious Disease Society of America (Griffith et al 2007), recommends prolonged treatment with 3 or 4 antimicrobial agents in combination for up to 18 months, based on pathogen- and patient-specific factors. Suggested agents for MAC-PD include clarithromycin or azithromycin, rifampin or rifabutin and ethambutol, and IV streptomycin or amikacin. Among these agents, the macrolide component appears to be particularly important, as emergence of resistance to clarithromycin or azithromycin is a risk factor for disease progression and mortality. This complex multidrug therapy is often complicated by side effects and/or drug interactions that require discontinuation of one or more of the agents and predisposes patients to treatment failure and development of drug-resistant NTM, further complicating therapy. Importantly, none of the guideline-based therapeutic options, as individual drugs or combination regimens, have been evaluated for safety and efficacy under controlled conditions in prospectively designed clinical studies in patients with NTM-PD and none are specifically approved for such use.

In addition to complex antimicrobial therapy, patients diagnosed with NTM-PD are required to undertake aggressive daily respiratory hygiene regimens including chest physiotherapy and nebulizer treatments in order to clear copious pulmonary secretions and prevent atelectasis. These adjunctive therapies are time-consuming and can have an adverse impact on patients' quality of life (CDER 2016). Although up to 86% of patients with pulmonary disease due to MAC may achieve sputum conversion (from positive to negative) on therapy, the translation of microbiological sputum clearance to immediate or long-term clinical outcomes is unclear since controlled and prospectively designed clinical studies to assess the link are lacking. Once established, NTM-PD can cause progressive destruction of the pulmonary parenchyma, and, despite prolonged therapy, outcomes remain unsatisfactory, with high rates of reinfection and mortality. Mortality rates due to NTM-PD range from 25% to 40% at 5 years. The main factors of poor outcomes identified in mortality studies at 5 years corresponded to an advanced age, the existence of respiratory comorbidities, radiological cavity lesions, and specific mycobacterial species, including Mycobacterium xenopi. The high rate of intrinsic resistance of NTM species to conventional antibiotics and antimycobacterial agents, as well as the poor tolerability exhibited to agents utilized in current SOC regimens, contributes to relatively high mortality rates for NTM-PD. The development and approval of novel drugs with demonstrated efficacy and improved tolerability for NTM-PD is thus a critical research priority.

SPR720, the phosphate pro-drug of SPR719, is a novel aminobenzimidazole bacterial DNA gyrase (GyrB) inhibitor. SPR719 has broad-spectrum activity against clinically relevant mycobacteria in vitro and in vivo, in hollow fiber (HF) infection models. Antibacterial agents amendable to safe and efficacious dosing with a single daily dosage are particularly desirable.

### SUMMARY

The present disclose provides methods of treating NTM-PD and other bacterial infections, including pulmonary tuberculous. Clinical studies have established the safety of SPR720 up to daily oral dosages of 2000 mg and that once daily oral dosing is sufficient to provide efficacious plasma levels of the active agent, SPR719. Clinical studies have also shown that there is no significant food effect for SPR720 and that the drug may be given in to a patient in the fed or fasted state. Clinical studies have also established that SPR720 can be administered safely to elderly patients. The present disclosure provides a method of treating a bacterial infection in a patient comprising administering a daily oral dose of SPR720, or a pharmaceutically acceptable salt thereof, the oral dose comprising 100 mg SPR720 to 1500 mg SPR720. In an embodiment the patient is an adult human patient. In an embodiment the daily oral SPR720 comprises 500 mg SPR 720 to 1000 mg SPR720, or 500 mg SPR720, or 1000 mg SPR720. SPR720 can be administered to a patient in the fed or fasted state. The disclosure provides methods of treating tuberculosis infections and nontuberculous mycobacteria infections. The nontuberculous mycobacteria infection can be nontuberculous mycobacteria pulmonary disease (NTM-PD) or treatment refractory NTM-PD. The nontuberculous mycobacteria infection can be due to Mycobacterium avium complex (MAC) infection. SPR720 or a pharmaceutically acceptable salt thereof can be administered to a patient for a period of 7 days to 28 days. In an embodiment, a daily dose of 500 mg or 1000 mg SPR720 is administered to the patient for this period of time as a single oral daily dose. SPR720 or pharmaceutically acceptable salt thereof can be administered alone or in combination with an additional antibacterial agent. The additional antibacterial agent can be, for example, clarithromycin, ethambutol hydrochloride, azithromycin, rifampin, and rifabutin.

The disclosure provides a method of treating a condition in a patient caused or exacerbated by a nontuberculous mycobacteria infection comprising administering a once daily oral dosage comprising SPR720 or a pharmaceutically acceptable salt thereof to the patient, wherein the patient is an adult human patient, the oral dosage contains 500 mg SPR720 or 1000 mg SPR720, and the once daily oral dosage is administered for 7 days to 28 days.

The disclosure provides a method of treating nontuberculous mycobacteria pulmonary disease (NTM-PD) in a patient, comprising orally administering to the patient 500 mg SPR720 once daily for at least 14 days or 1000 mg SPR720 once daily for at least 14 days.

The disclosure also provides administering 500 mg SPR720 once daily for at least 28 days or administering 1000 mg SPR720 once daily for at least 28 days.

The patient may have a Mycobacterium avium complex (MAC) infection.

The MAC infection includes infection with one or both of M. abscessus and M. Kansas.

The patient may have an underlying condition such as immunodeficiency, chronic obstructive pulmonary disease (COPD), cystic fibrosis, cavitary or fibro-cavitary lung disease, current or previous treatment with an antitumor necrosis factor agent, neutropenia, a hematologic malignancy, a history of hematopoietic stem cell transplant, a history of solid organ transplant, current treatment with immunosuppressive therapy, current treatment with cancer chemotherapy, current treatment with monoclonal antibodies for autoimmune disease, previous or current C. difficile infection, hepatic impairment as evidenced by alanine aminotransferase or aspartate aminotransferase >2× upper limit of normal (ULN) or total bilirubin >1.5×ULN, clinical signs of cirrhosis or end-stage hepatic disease (e.g., ascites, hepatic encephalopathy), renal impairment as evidenced by a creatine clearance of <50 mL/min., and end-stage renal disease requiring hemodialysis or peritoneal dialysis.

Treatment with SPR720 may be continued for at least 14 days, at least 21, days, at least 28 days, or at least until the patient experiences improvement in at least one of the following symptoms: cough, hemoptysis, fatigue, chest pain, poor appetite, fever, night sweats, and weight loss.

The patient may be treated only with SPR720 or may be treated with one or more additional drugs, such as another infective compound or another drug to improve lung function. For example, the patient may be treated with a combination of SPR720 and an agent selected from amikacin,-azithromycin, rifampin, rifabutin, ethambutol HCl, clarithromycin, streptomycin, amikacin, or an inhaled steroid.

The NTM-PD can be nodular bronchiectatic NTM-PD or fibro-cavitary NTM-PD.

The disclosure also provides a method of treating a patient who has a nontuberculous mycobacterial infection comprising:
(i) determining that the nontuberculous mycobacterial infection is resistant to at least one of amikacin, clarithromycin, and fluoroquinolone antibiotics; and
(ii) orally administering to the patient 500 mg SPR720 once daily for at least 14 days or 1000 mg SPR720 once daily for at least 14 days.

### BRIEF DESCRIPTION OF THE DRAWINGS

FIG. 1. Study design for Single Ascending Dose (SAD) and Multiple Ascending Dose (MAD) studies.

FIGS. 2A and 2B. Geometric mean plasma SPR719 concentration-time curves Following SPR720 administration. FIG. 2A. Single-ascending doses (SAD) of SPR720 administered in the fed and fasted state and FIG. 2B. SPR720 1000 mg dose administered in the fed and fasting states.

FIGS. 3A, 3B, and 3C. Geometric mean plasma SPR719 concentration-time curves following multiple ascending dose administration of SPR720 over 7 and 14 days. FIG. 3A. Day 1, FIG. 3B, Day 7, and FIG. 3C, Day 14.

FIG. 4. The incidence of adverse events recorded during the SAD study (Table 1).

FIG. 5. The incidence of adverse events recorded in treated cohorts during the MAD study (Table 2).

FIG. 6. Schematic of efficacy study of SPR720 for NTM-PD due to Mycobacterium Avium Complex (MAC). ABBREVIATIONS: DB=Double-Blind, EOT=End of Treatment, OL=Open Label, QD=Once daily, SOC=Standard of Care.

### DETAILED DESCRIPTION

### Terminology

Prior to describing the disclosure in detail, the following terms may be helpful. Unless otherwise specified all terms carry their ordinary meaning, accepted in the art of pharmaceutical formulations or methods of treating bacterial infections in patients.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or language indicating an example (e.g., “such as”), is intended merely for illustration and does not pose a limitation on the scope of the invention unless otherwise claimed. No language in the specification should be construed as indicating any non-claimed element as essential to the practice of the invention.

The terms “a” and “an” do not denote a limitation of quantity, but rather denote the presence of at least one of the referenced items.

The term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 10%, which are also effective and safe are included in the value. Thus, compositions slightly outside the cited ranges are also encompassed by the scope of the present claims.

The terms “comprising,” “including,” and “containing” are non-limiting. Other non-recited elements may be present in embodiments claimed by these transitional phrases. Where “comprising,” “containing,” or “including” are used as transitional phrases other elements may be included and still form an embodiment within the scope of the claim. The open-ended transitional phrase “comprising” encompasses the intermediate transitional phrase “consisting essentially of” and the close-ended phrase “consisting of.”

“Administering” includes any pharmaceutically acceptable mode of administration, such as oral, intravenous, intramuscular injection, subcutaneous injection, transdermal, transmucosal, buccal, or oral inhaled administration.

“Treatment,” as used herein includes providing the SPR720 sufficient to: (a) reduce probability a disease or a symptom of a disease from occurring in a patient who is be predisposed to the disease but has not yet been diagnosed as having it (e.g. prevent a mycobacterial bacterial infection in a patient traveling to an area where risk of exposure to bacterial infection is high); (b) inhibiting the disease, i.e. arresting its development; and (c) relieving the disease, i.e., causing regression of the disease. “Treating” and “treatment” also means providing a therapeutically effective amount of SPR720 and optionally one or more additional active agents to a patient having a MAC resistant bacterial infection or an antibiotic resistant mycobacterial infection.

A “therapeutically effective amount” of a pharmaceutical combination of this disclosure means an amount effective, when administered to a patient, to provide a therapeutic benefit such as an amelioration of symptoms, e.g., an amount effective to decrease the symptoms of a bacterial infection. For example, a patient infected with a bacterial infection may present abnormal levels of certain blood cells, especially leukocytes (white blood cells) for example, an increase in neutrophils and a decrease in lymphocytes. A therapeutically effect amount is thus an amount sufficient to provide a return of leukocyte levels to the normal range. A therapeutically effective amount is also an amount sufficient to prevent a significant increase or significantly reduce the detectable level of mycobacteria or mycobacterial antibodies in the patient's blood, serum, or tissues.

When a weight of SPR720 that may be in a salt form is given the value refers to the amount of SPR720 rather than the weight of SPR720 salt. Unless clearly contraindicated by the context, “SPR720” includes SPR720 in free phosphate form or as a pharmaceutically acceptable salt or hydrate and can be in an amorphous solid or crystalline form.

SPR719 (also known as VXc-486, CAS Reg. No. 1384984-18-2) is a potent antibacterial agent, with gyrase B/Par E inhibitory activity, having the following structure:

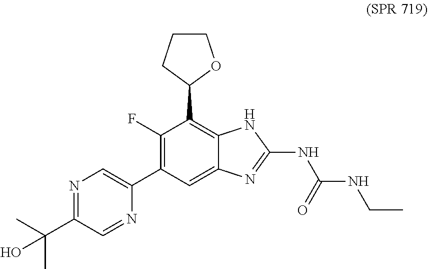 [SMILES: CCNC(=O)Nc1nc2cc(-c3cnc(C(C)(C)O)cn3)c(F)c([C@H]3CCCO3)c2[nH]1]

SPR720 (CAS Reg. No. 1384984-31-9), generic name fobrepodacin, is an orally active phosphate prodrug of SPR719 having the structure:

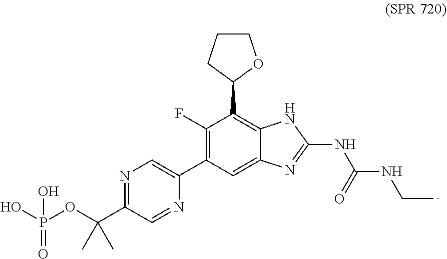 [SMILES: CCNC(=O)Nc1nc2cc(-c3cnc(C(C)(C)OP(=O)(O)O)cn3)c(F)c([C@H]3CCCO3)c2[nH]1]

### Pharmaceutical Compositions

“Pharmaceutical compositions” are compositions comprising at least one active agent, such as a SPR720, and at least one other substance, such as a carrier. Pharmaceutical compositions meet the U.S. FDA's GMP (good manufacturing practice) standards for human or non-human drugs. The term “carrier” applied to pharmaceutical compositions/combinations of the disclosure refers to a diluent, excipient, or vehicle with which an active compound is provided.

Pharmaceutical compositions of the disclosure include any pharmaceutically acceptable formulations capable of delivering systemic SPR719 to the patient. Examples include oral, nasal, transdermal, sublingual, rectal, intravenous (both bolus and infusion), inhalable, and injection (intraperitoneal, subcutaneous, intramuscular, or parenteral) formulations. Oral formulations are particularly contemplated. Oral formulation may be in a dosage unit such as a tablet, pill, capsule, powder, granule, liquid, syrup, emulsion, or droplet.

The dosage form containing the SPR720 contains an amount sufficient to provide a therapeutic effect by the chosen route of administration. The composition may contain from about 2,000 mg to about 50 mg (preferably, from about 1,000 mg to about 100 mg) of SPR720 or salt form thereof (weight is given for the free base form) and may be constituted into any form suitable for the selected mode of administration. The dosage form may be formulated for immediate release or controlled release, including delayed release or sustained release. An SPR720 dosage form can include SPR719 as the only active agent or can be formulated in combination with one or more additional active agents, such as another antibacterial agent. Suitable additional active agents include standard of care agents for treating mycobacterial infections, such as clarithromycin (e.g. 500 mg to 1000 mg daily dose) ethambutol hydrochloride (e.g., approximately 15 mg/kg daily dose), azithromycin (e.g., 250 mg to 500 mg daily dose), streptomycin (no more than 2 grams daily, oral or IM), rifampin (e.g., 100 mg to 1000 mg daily dose or 600 mg daily dose), rifabutin (e.g., 100 mg to 500 mg daily dose or 300 mg daily dose), isoniazid (100 mg to 1000 mg daily dose, or 200 mg, 300 mg, 400 mg, or 500 mg daily dose), or pyrazinamide (100 mg to 1000 mg daily dose, or 200 mg, 300 mg, 400 mg, 500 mg, 600 mg, or 700 mg daily dose), ethionamide (100 mg to 500 mg daily dose, or 200 mg, 250 mg, 300 mg, or 350 mg daily dose), clofazimine, inhaled amikacin, or bedaquiline.

### Methods of Treatment

The disclosure includes methods of treating bacterial infections, such as tuberculosis and non-tubercular mycobacterial infections. Non-tubercular mycobacterial infections that may be treated with SPR720/SPR719 include infections due to Mycobacterium avium complex (MAC), which includes M. avium, M. intracellulare, M. chimaera and several subspecies. Other non-tubercular mycobacterial infections that may be treated with SPR720/SPR719 include photochromogens such as M. kansasii, M. simiae, and M. marinum; scotochromogens such as M. scrofulaceum, M. parascrofulaceum and M. szulgai; nonchromogens, a group that includes MAC, as well as M. ulcerans, M. xenopi, M. malmoense, M. terrae, M. haeomphilium, and M. genavense; and rapid growing nonchromogenic species such as M. chelonae, M. chelonae-abscessus, M. fortuitum, and M. peregrinum; and other non-tubercular mycobacterial organisms, such as M. smegmatis, M. paratuberculosis, M. marinum, M. simiae, and M. flavescens.

NTMs are responsible for a number of conditions. This disclosure includes a method of treating a condition caused by NTM infection comprising administering a SPR720 or salt thereof, where SPR720 the only active agent or is administered in combination with another active agent, to a patient having such a condition. This disclosure also includes a method of treating a condition exacerbated by NTM infection. Conditions than can be due to NTM infection or exacerbated by NTM infection include respiratory infection, lung infections, Johne's disease (in ruminants), Crohn's disease, osteomyelitis, peritonitis, pyelonephritis, cervical lymphadenitis, disseminated infection in immunocompromised patients, pulmonary disease, disseminated NTM infection, extrapulmonary NTM, and refractory NTM infection.

A “therapeutically effective amount of a pharmaceutical composition” is an amount effective, when administered to a subject, to provide a therapeutic benefit, such as to decrease the morbidity and mortality associated with bacterial infection and/or effect a cure. In certain circumstances a subject suffering from a bacterial infection may not present symptoms of being infected. Thus, a therapeutically effective amount of a compound is also an amount sufficient to significantly reduce the detectable level of microorganism in the subject's blood, serum, sputum, or other bodily fluids, or tissues. A therapeutically effective amount of SPR720 can also be an amount sufficient to reduce the clinical symptoms of a bacterial infection or mycobacteria infection.

Individuals with NTM infections have considerable variability in their clinical presentation. Diagnosis can be made from clinical symptoms or via culture of respiratory fluid, sputum, or mucous. The symptoms of mycobacteria infection include chronic cough, fatigue, frequent throat clearing, shortness of breath (dyspnea), excessive mucus (sputum) production, fever, night sweats, loss of appetite, unintended weight loss, wheezing, and chest pain. The disclosure also includes, in certain embodiments, using compounds of the disclosure in prophylactic treatment and therapeutic treatment. A therapeutically effective amount of SPR720 includes an oral dosage sufficient to relieve or reduce one or more clinical symptoms of NTM infection.

In the context of prophylactic or preventative treatment, a “therapeutically effective amount” is an amount sufficient to significantly decrease the incidence of or morbidity and mortality associated with bacterial infection. For example, prophylactic treatment may be administered when a subject is known to be at enhanced risk of bacterial infection, such as a cystic fibrosis or ventilator patient. A significant reduction is any detectable negative change that is statistically significant in a standard parametric test of statistical significance such as Student's T-test, where p<0.05.

Additional indicators of infective treatment include fewer colony forming units (CFU)/mL in sputum relative after treatment to the number of CFU/mL in sputum from a patient prior to SPR720 treatment.

A “patient” is any individual that can benefit from treatment with SPR720. Patients include adult human patients and pediatric and infant patients. Patients include human and non-human patients. For examples a patient can be a livestock animal such as a cow, pig, sheep, horse, or goat or a companion animal such as a dog or cat. Dosages of SPR720 are given for adult human patients unless otherwise indicated.

SPR720 may be administered on any dosage schedule that provides a therapeutically effective amount of SPR720 to the patient. For example, SPR720 can be administered 1, 2, 3, or 4 times per day. An embodiment includes once daily oral administration for a period of one or more days. For example, SPR720 can be administered one or two times daily, or once daily, for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. SPR720 can be orally administered for one or two times daily, or one time daily for a period of 1 month, 2 month, or 3 months. In an embodiment, SPR720 is administered once daily for a period of 7 days, 14 days, or 28 days, 1 month, 2 months, 3 months, 4 months, 5 months, 6 months, or up to 12 months.

SPR720 can be administered via any acceptable route for capable of providing therapeutically effective plasma levels of SPR719 in the patient. Examples include oral, nasal, transdermal, sublingual, rectal, intravenous (both bolus and infusion), inhalable, and injection (intraperitoneal, subcutaneous, intramuscular, or parenteral) administration. Oral administration is particularly contemplated. Oral administration of SPR720 can be effected by any pharmaceutically acceptable oral dosage form, such as a tablet, pill, capsule, powder, granule, liquid, syrup, emulsion, or droplet.

SPR720 can be administered in any amount capable of providing a safe and therapeutically effective amount of SPR719 to the patient. For example, a daily dose of from about 2,000 mg to about 50 mg (preferably, from about 1,000 mg to about 100 mg) of SPR720 or salt form thereof (weight is given for the free base form) can be administered to an adult human patient. For example, 100 mg, 200 mg, 250 mg, 300 mg, 400 mg, 500 mg, 600 mg, 700 mg, 750 mg, 800 mg, 900 mg, 1000, 1100 mg, 1200 mg, or 1250 mg SPR720 can be administered daily.

SPR720 or a pharmaceutically acceptable salt thereof can be administered alone to provide SPR719 as the only active agent or SPR720 can be administered in combination with one or more additional active agents, such as an additional antibacterial agent. For an adult human patient, a method of treatment in which an oral dose of 500 mg or 1000 mg SPR720 is administered for a period of 7 to 28 days is included as an embodiment of this disclosure. Pediatric patients can also be treated with SPR720. The pediatric dose can be reduced relative to the adult dose to account for the lower weight of the pediatric patient.

There is only one approved method for treating NTM infection, amikacin liposome inhalation suspension. SPR720 can be administered in combination with amikacin liposome inhalation suspension. SPR720 can be administered to a patient who has been treated with amikacin liposome inhalation suspension for a period of 1 or more months, for example 6 weeks, 6 months, or 12 months, without improvement or whose symptoms have not been adequately addressed. Methods of treatment include administering SPR720 in combination with 1 or 2 additional active agents for a period of up to 18 months. Suitable additional active agents include standard of care agents for treating mycobacterial infections, such as clarithromycin (e.g. 500 mg to 1000 mg daily dose) ethambutol hydrochloride (e.g., approximately 15 mg/kg daily dose), azithromycin (e.g., 250 mg to 500 mg daily dose), streptomycin (no more than 2 grams daily, oral or IM), rifampin (e.g., 100 mg to 1000 mg daily dose or 600 mg daily dose), rifabutin (e.g., 100 mg to 500 mg daily dose or 300 mg daily dose), isoniazid (100 mg to 1000 mg daily dose, or 200 mg, 300 mg, 400 mg, or 500 mg daily dose), or pyrazinamide (100 mg to 1000 mg daily dose, or 200 mg, 300 mg, 400 mg, 500 mg, 600 mg, or 700 mg daily dose), ethionamide (100 mg to 500 mg daily dose, or 200 mg, 250 mg, 300 mg, or 350 mg daily dose), clofazimine, inhaled amikacin, or bedaquiline.

It is recommended that SPR720 not be administered in combination with a strong inhibitor or inducer of CYP3A. Methods of treatment include advising the patient that SPR720 should not be administered in combination with a strong CYP3A inhibitor or inducer, and if the patient is currently taking a strong CYP3A inducer or inhibitor switching the patient to a different medication prior to administering SPR720, temporarily discontinuing the strong CYP3A inhibitor or inducer, or administering the strong CYP3A inhibitor or inducer medication in combination with SPR720 and monitoring the patient's SPR719 plasma levels weekly and reducing or increasing the dose of SPR720 so that the patient's SPR719 plasma levels remain between 100 ng/nl and 10000 ng/nl.

### EXAMPLES

### Example 1. Pharmacokinetics of SPR720/SPR719 Administered in the Fed and Fasted States in Single Ascending Dose Study

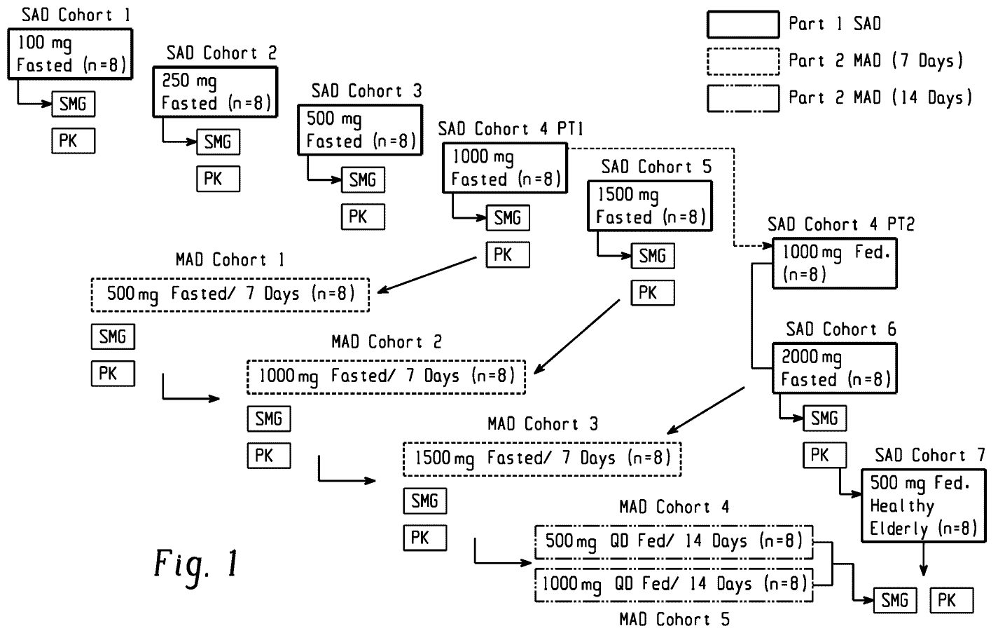

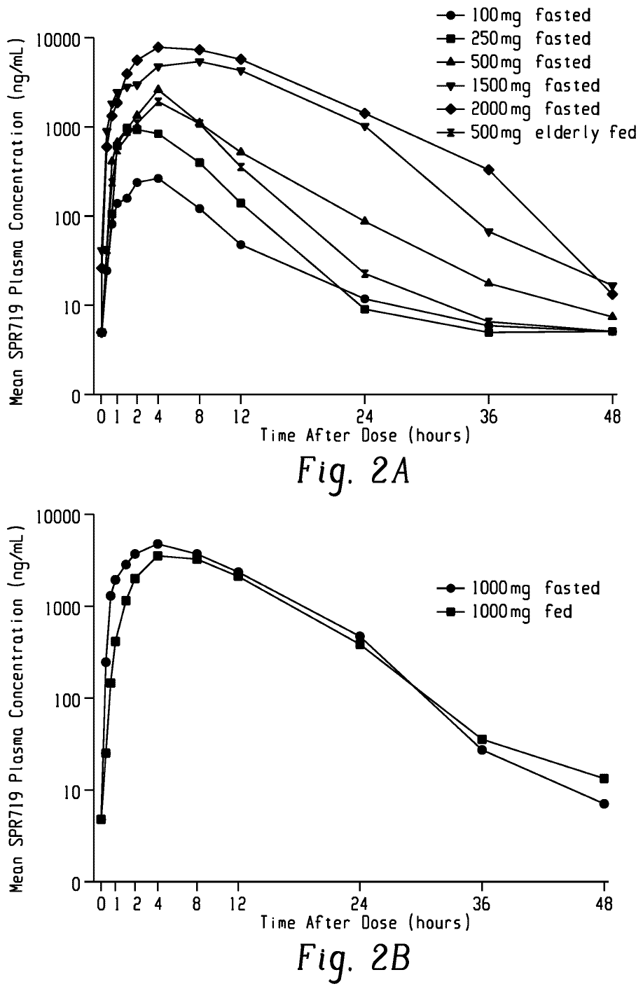

The following study was conducted in healthy volunteers to evaluate the safety, tolerability, and pharmacokinetics of SPR720/SPR719. The study included 7 SAD cohorts, of which one was a food effect cohort. SPR720 or placebo (n=8/cohort, 3:1 randomization) were administered to subjects at doses of 100 to 2000 mg in the fasted or fed state. The cohorts administered SPR720 in the fed state were administered 1000 mg or 500 mg (healthy elderly cohort) SPR720. A schematic of the study design appears in FIG. 1.

Across SAD cohorts, a dose proportional and greater-than dose proportional increase in plasma SPR719 Cₘₐₓ and AUC₀₋₂₄, was observed. Following administration of a high fat meal, a small decrease in plasma exposure relative to plasma exposure in the fasted state. This decrease was considered non-clinically significant. The median Tₘₐₓ for SPR719 ranged from 2.75 hours to 8 hours across cohorts and the mean elimination half-life (t_{1/2}) ranged from 2.92 to 4.5 hrs. Urinary excretion of SPR719 (0-24 hrs.) was low (0.5%). Mean plasma concentration/time curves for fasted and elderly fed cohorts are provided in FIG. 1A and for fed and fasted cohorts each administered 1000 mg SPR720 are provided in FIG. 1B. Treatment adverse events for all SAD cohorts are listed in Table. 1.

TABLE 1
Incidence of treatment-emergent adverse
events in single-ascending dose cohorts

| | Number (%) of Subjects | | | | |
|---|---|---|---|---|---|
| | | | | | Cohort 4 |
| | Placebo | Cohort 1 | Cohort 2 | Cohort 3 | 1000 mg |
| SAD | Overall | 100 mg | 250 mg | 500 mg | Fasted |
| Event | (n = 14) | (N = 6) | (N = 6) | (N = 6) | (N = 6) |
| Nausea | 0 | 0 | 0 | 0 | 0 |
| Headache | 1 (7.1) | 0 | 0 | 0 | 1 (16.7) |
| Vomiting | 0 | 0 | 0 | 0 | 0 |
| Diarrhea | 0 | 0 | 0 | 1 (16.7) | 0 |

| | Cohort 4 | | | | |
|---|---|---|---|---|---|
| | 1000 mg | Cohort 5 | Cohort 6 | 500 mg | Overall |
| | Fed | 1500 mg | 2000 mg | Elderly | SPR720 |
| Event | (N = 6) | (N = 6) | (N = 6) | (N = 6) | (n = 42) |
| Nausea | 0 | 4 (66.7) | 5 (83.3) | 0 | 9 (21.4) |
| Headache | 0 | 3 (50.0) | 3 (50.0) | 0 | 7 (16.7) |
| Vomiting | 0 | 3 (50.0) | 5 (83.3) | 0 | 8 (19.0) |
| Diarrhea | 0 | 0 | 2 (33.3) | 0 | 3 (7.1) |

Of 42 subjects, 18 (42.9%) reported a total of 35 TEAEs; all were mild or moderate. More subjects reported TEAEs in the 1500 mg and 2000 mg cohorts (66.7% and 100%, respectively)

### Example 2. Pharmacokinetics of SPR720/SPR719 Administered in the Fed and Fasted States in Multiple Ascending Dose Study

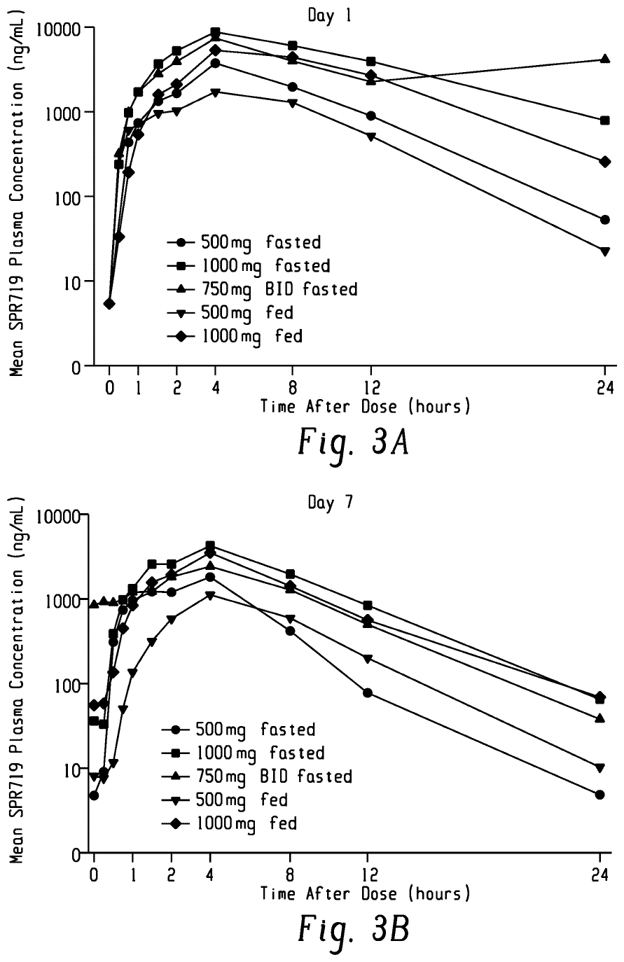

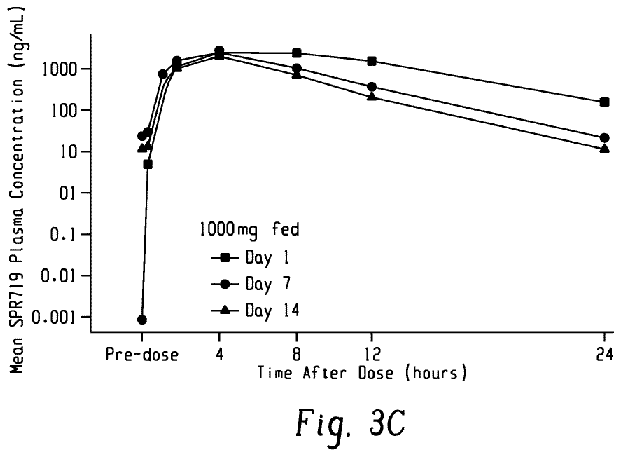

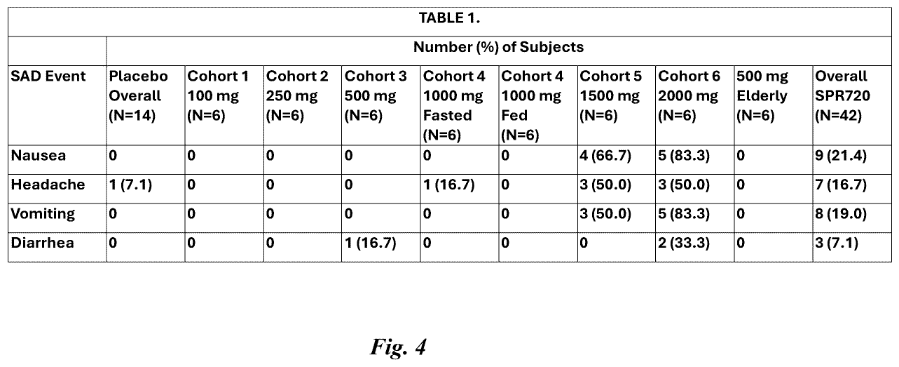

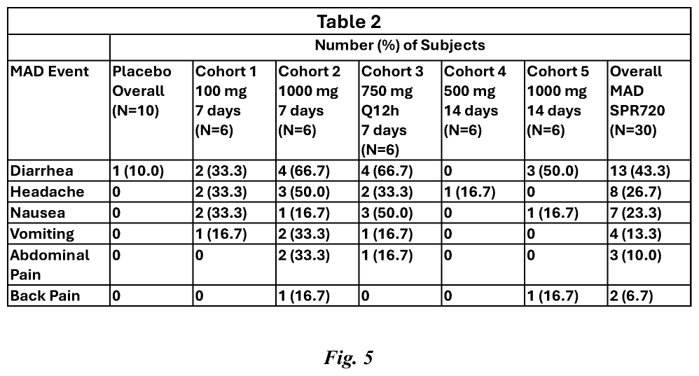

The multiple ascending dose study utilized 5 cohorts of 8 subjects. Three cohorts were administered a total daily dose of 500 mg to 1500 mg in the fasted state for 7 days. Two cohorts were administered a total daily dose of 500 mg or 1000 mg in the fed state for 14 days. Safety was also monitored throughout the study. Across MAD cohorts, SPR719 both plasma Cₘₐₓ and AUC increased in a greater than dose proportional manner with increasing oral doses of SPR720. Repeated oral dosing of SPR720 in healthy human subject resulted in a decrease (˜40%) in plasm exposure of SPR719 at day 7 relative to day 1, suggesting induction of elimination pathways for APR719. Plasma AUC₀₋₂₄ was similar at Days 7 and 14, indicating that induction of elimination had stabilized by Days 7-14. This conclusion was further supported by stable SPR719 trough concentrations by Day 7 (data not shown). Mean plasma concentration/time curves are shown for the MAD cohorts at days 1 (FIG. 3A), 7 (FIG. 3B), and 14 (FIG. 3C). Treatment adverse events for all SAD cohorts are listed in Table. 2.

TABLE 2
Incidence of treatment-emergent adverse events in multiple-ascending dose cohorts

| | MAD | | | | | | |
|---|---|---|---|---|---|---|---|
| | Number (%) of Subjects | | | | | | |
| | | | | Cohort 3 | | | |
| | | Cohort 1 | Cohort 2 | 750 mg | Cohort 4 | Cohort 5 | Overall |
| | Placebo | 500 mg | 1000 mg | q12 h | 500 mg | 1000 mg | MAD |
| | Overall | 7 days | 7 days | 7 days | 14 days | 14 days | SPR720 |
| Event | (N = 10) | (N = 6) | (N = 6) | (N = 6) | (N = 6) | (N = 6) | (N = 30) |
| Diarrhea | 1 (10.0) | 2 (33.3) | 4 (66.7) | 4 (66.7) | 0 | 3 (50.0) | 13 (42.3) |
| Headache | 0 | 2 (33.3) | 3 (50.0) | 2 (33.3) | 1 (16.7) | 0 | 8 (26.7) |
| Nausea | 0 | 2 (33.3) | 1 (16.7) | 3 (50.0) | 0 | 1 (16.7) | 7 (23.3) |
| Vomiting | 0 | 1 (16.7) | 2 (33.3) | 1 (16.7) | 0 | 0 | 4 (13.3) |
| Diarrhea | 0 | 0 | 2 (33.3) | 1 (16.7) | 0 | 0 | 3 (10.0) |
| Abdominal | 0 | 0 | 1 (16.7) | 0 | 0 | 1 (16.7) | 2 (6.7) |
| Pain | | | | | | | |

No serious adverse events were reported in either the SAD or MAD study.

Of 30 subjects who received SPR720, 18 (60.0%) reported a total of 101 TEAEs; all were mild or moderate severity. More subjects reported TEAEs in the 1000 mg or 1500 mg (750 mg q12h) cohorts (50.0% and 66.7%, respectively). One subject in MAD cohort 3 (750 mg q12h) discontinued study drug due to increased pancreatic enzymes; this was asymptomatic and resolved without interventional treatment. This could be attributed to persistently high trough plasma concentrations of SPR719>1000 ng/mL throughout the dosing period. Slight elevations in ALT (<3×ULN) were observed over 14 days of dosing, which were asymptomatic and reversible; there were no cases of Hy's Law. SPR720 was well-tolerated at repeat daily oral doses up to 1000 mg over the maximum duration of 14 days. In SAD cohorts, Cₘₐₓ and AUC increased in a dose-proportional and greater than dose-proportional manner, respectively. In MAD cohorts, exposure declined between Days 1 and 7, but was similar at Days 7 and 14; urinary excretion of SPR719 was minimal. Together with HF pharmacodynamic data, the human safety and PK data for SPR720 suggest that predicted therapeutic exposures can be attained with a well-tolerated once-daily dose.

### Example 3. Efficacy of SPR720 for NTM PD Due to Mycobacterium Avium Complex (MAC)

This example is a randomized, partially blinded, placebo- and comparator-controlled study to evaluate the safety, tolerability, PK, and efficacy of 2 dose levels of SPR720 compared with placebo or SOC for the treatment of patients with non-Tubercular Mycobacterial infection, Pulmonary Disease (NTM-PD) due to Mycobacterium avium complex (MAC). The SPR720 and placebo arms are double blinded, and the SOC arm is open label. Approximately 90 male and female patients 18 years of age and older with a diagnosis of NTM-PD due to MAC are treated for 28 days. Patients who had received prior treatment were eligible if they had culture evidence of persistent, recurrent, or relapsed disease; had been off therapy for at least 6 months; and are likely to reinitiate or resume treatment.

The study includes 4 treatment arms.

Treatment Arm 1: SPR720 Low Dose: Double-blinded SPR720 investigational product (IP) 500 mg (2 capsules of 250 mg SPR720 and 2 capsules of placebo) po once daily for 28 days.

Treatment Arm 2: SPR720 High Dose: Double-blinded SPR720 IP 1000 mg (4 capsules of 250 mg SPR720) once daily for 28 days.

Treatment Arm 3: Placebo: Double-blinded placebo (4 capsules of placebo) once daily for 28 days.

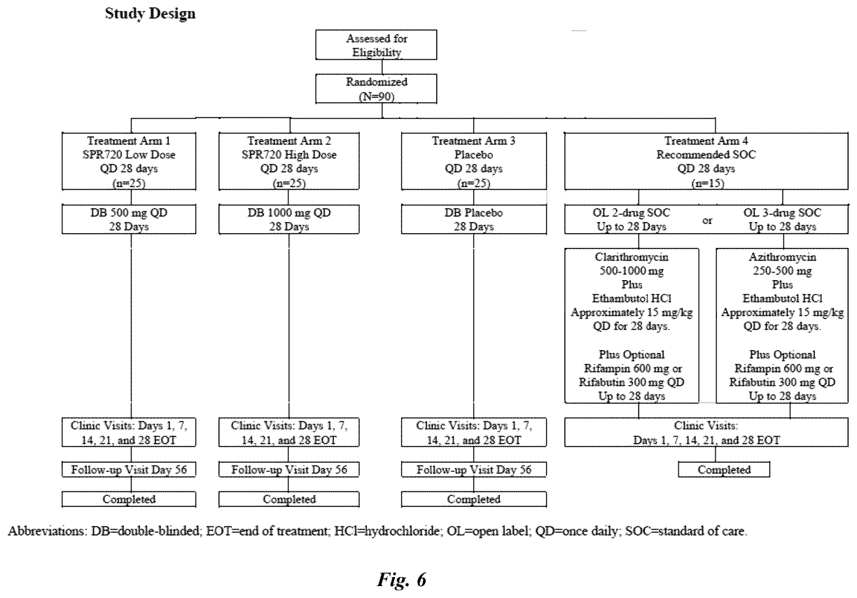

Treatment Arm 4: SOC: At Investigator's discretion. Recommended as follows: 2-drug or 3-drug SOC, including clarithromycin 500 to 1000 mg PLUS ethambutol hydrochloride (HCl) approximately 15 mg/kg or azithromycin 250 to 500 mg once daily PLUS ethambutol HCl approximately 15 mg/kg once daily for 28 days. Optional rifampin 600 mg or rifabutin 300 mg once daily for up to 28 days may be added to the SOC regimen. A schematic of this study is provided at FIG. 6.

Randomization is stratified by diagnosis at enrollment as either 1) nodular bronchiectatic or 2) fibro-cavitary NTM-PD. Clinic study visits occur on Days 1, 7, 14, 21, and 28 (end-of-treatment [EOT] visit) (±1 day). Patients in the double-blinded Treatment Arms 1, 2, and 3 are evaluated at a follow-up-visit on Day 56 (±2 days). Patients in the open-label SOC Treatment Arm 4 are discontinued from the study after the EOT visit.

Patient-collected pooled expectorated sputum is collected on Days 1, 6, 13, 20, and 27 (i.e., the day before clinical study visits over an approximate 24-hour (h) period) for all patients and, for patients in Treatment Arms 1, 2, and 3, on Day 55 (i.e., the day before the follow-up visit on Day 56). In all cases, the patient-collected sputum is collected on the day before the actual clinic visit. Sputum is also to be collected at each clinic visit using a standard induction protocol. Patients in Treatment Arms 1, 2, and 3 participate in the collection of blood samples for PK analysis as follows:
(i) At least 3 sites, each enrolling at least 18 patients (at least 6 per treatment arm) are used for intensive PK evaluation. On Days 1 and 28, blood samples for intense PK are collected at pre-dose and postdose: 1 h, 2 h, 4 h, 8 h, 12 h, and 24 h. On Days 7, 14, and 21, blood samples are obtained at: pre-dose, 1 h post-dose, and just before discharge from the clinic.
(ii) At all remaining sites, samples are collected for sparse PK evaluation on Days 1, 7, 14, 21, and 28. Blood samples are obtained at pre-dose, 1 h after the oral dose, and just before discharge from the clinic.
(iii) Patients randomized to the SOC arm will not participate in PK collection. Actual dates and times of blood sampling and dates and times of study drug administration will be recorded.

Microbiological Intent-to-Treat Population. The micro-ITT Population includes randomized patients with a confirmed diagnosis of NTM-PD due to MAC and an induced sputum quantitative culture at baseline (Day 1) for MAC. Quantitative culture will be on solid agar (elective Middlebrook 7H10 medium to record CFUs) or, if quantitative culture result at baseline is not available, derived from positive growth on MGIT (Mycobacteria Growth Indicator Tube). Patients with a baseline culture growth of a species of Mycobacterium other than MAC will be excluded from the micro-ITT Population. Patients will be summarized by the treatment to which they were randomly assigned.

Pharmacokinetic Analysis Population. The PK Population includes all patients treated with at least 1 dose of SPR720 and who have at least 1 analyzable plasma PK sample.

Inclusion Criteria. The Inclusion Criteria for patients in the NTM PD study are as follows.

Patients must meet all the following inclusion criteria to be admitted to the study: 1. Provided written informed consent, 2. Male or female, 18 years of age or older at the time of consent, 3. Has a diagnosis of NTM-PD due to MAC 4. Had at least 1 prior positive culture (sputum or bronchoalveolar lavage) for MAC in the previous 6 months 5. Has an induced sputum culture at screening positive for MAC by at least one of the following methods performed by the microbiology laboratory quantitative culture on solid agar or growth on liquid media (MGIT), 6. Is either treatment naïve and has not received any prior treatment for MAC, OR if previously treated for MAC, has culture evidence of persistent, recurrent, or relapsed disease and has been off therapy for at least 6 months, 7. In the opinion of the Investigator, is ready to initiate treatment (treatment naïve) or reinitiate treatment (previously treated) within the next 3 months, and for whom a delay, in order to participate in a placebo-controlled clinical trial, is considered reasonable and clinically acceptable, 8. Had clinical signs and symptoms within the 6 weeks prior to consent that are consistent with NTM-PD with at least two of the following: a. chronic cough, b. fatigue, c. frequent throat clearing, d. shortness of breath (dyspnea), c. coughing up of blood (hemoptysis), f. excessive mucus (sputum) production, g. fever, h. night sweats, i. loss of appetite, j. unintended weight loss, k. wheezing, l. chest pain. 9. Has a measured forced expiratory volume in 1 second (% predicted FEV1)≥30% on pulmonary function test within 3 months prior to consent. 10. Has a chest radiograph (CXR) or computed tomography (CT) scan within 6 months prior to consent with findings consistent with NTM-PD. If no prior CXR or CT scan is available, a CXR or CT scan should be performed at screening to confirm eligibility. 11. If female, is of nonchildbearing potential (e.g., postmenopausal as demonstrated by follicle-stimulating hormone or surgical sterilization, i.e., tubal ligation or hysterectomy), or if of childbearing potential, is willing to commit to either sexual abstinence or use of at least 2 medically accepted, effective methods of birth control in combination (e.g., condom, spermicidal gel, oral contraceptive, indwelling intrauterine device, hormonal implant/patch, injections, approved cervical ring) from consent through follow-up. Patients randomized to the SOC arm will be followed until EOT. 12. If male, is willing not to donate sperm and, if engaging in sexual intercourse with a female partner who could become pregnant, willing to use a condom in addition to having his female partner use a highly effective method of birth control (such as an intrauterine device, diaphragm, oral contraceptives, injectable progesterone, subdermal implants, or a tubal ligation) from consent through follow-up. Patients randomized to the SOC arm will be followed up until EOT. 13. Is willing and able to comply with all study assessments and adhere to the protocol schedule.

Exclusion Criteria. Patients meeting any of the following exclusion criteria are not enrolled in the study: 1. In the opinion of the Investigator, is not a candidate for a 3-month delay in initiation of standard multidrug therapy in order to participate in a placebo controlled clinical trial or observation (e.g., severe symptoms, extensive disease burden). 2. Has disseminated or extrapulmonary NTM. 3. Has end-stage NTM-PD or treatment-refractory NTM-PD and is unlikely to respond to protocol-specified SOC treatment. 4. Had isolation on sputum cultures of any species of Mycobacterium other than a species included in MAC within the past 6 months. 5. Had prior isolation of MAC with macrolide resistance. 6. Has active tuberculosis at time of consent. 7. Has received any systemic (oral or intravenous) or inhaled antibiotic with activity against MAC between consent and randomization. 8. Has a potentially confounding underlying pulmonary disease, including but not limited to cystic fibrosis, active pulmonary (primary or metastatic), NTM-hypersensitivity disease pneumoconiosis, or another advanced lung disease with a % predicted FEV1 (Forced expiratory volume in 1 second)<30%. 9. Has a history of a positive test for HIV, known CD4 count <200/mm³ within the last year before consent, or a diagnosis of AIDS. 10. Has an immunodeficiency or an immunocompromised condition, including neutropenia (<1000 neutrophils/mm³ obtained from the central laboratory at screening), hematologic malignancy, a history of hematopoictic stem cell transplant, a history of solid organ transplant, is receiving immunosuppressive therapy (e.g., cancer chemotherapy, monoclonal antibodies for autoimmune disease, or medications to prevent transplant rejection), and has had long-term use of systemic corticosteroids (e.g., ≥20 mg/day of prednisone or systemic equivalent for at least 2 weeks). 11. Has a history of known or suspected C. diff infection. 12. Has a history of epilepsy or known seizure disorder (excluding a history of childhood febrile seizures). 13. Has hepatic impairment at screening, as evidenced by alanine aminotransferase or aspartate aminotransferase >2× upper limit of normal (ULN) or total bilirubin >1.5×ULN, or clinical signs of cirrhosis or endstage hepatic disease (e.g., ascites, hepatic encephalopathy). 14. Has renal impairment (creatinine clearance <50 mL/min) or end-stage renal disease requiring hemodialysis or peritoneal dialysis. 15. If female, is pregnant or breastfeeding. 16. Has a corrected QT (QTc) interval on electrocardiogram (ECG)>470 ms. 17. Has consumed drugs or supplements that are strong cytochrome P450 (CYP) 3A4 enzyme inducers or strong CYP3A4 inhibitors within 4 weeks of randomization. 18. Has consumed drugs or supplements that are substrates of the hepatic transporters OATP1B1 or OATP1B3 (see within 1 week of randomization). 19. Has a documented hypersensitivity reaction or anaphylaxis to SPR720 or any of the specified SOC medications. 20. Has received any investigational medication in the year before the time of consent. 21. Has any other condition or prior therapy, which would make the patient unsuitable for the study, including compliance with all study assessments and adherence to the protocol schedule.

Withdrawal from Study. A patient will be considered to have completed the study when he or she completes the EOT visit for patients in Treatment Arm 4 and the follow-up visit on Day 56 (±2 days) for patients in Treatment Arms 1, 2, and 3. Patients may withdraw from the study or be withdrawn at the request of the Investigator or sponsor at any time. Examples of reasons for study withdrawal include the following: (i) The patient withdraws consent or requests withdrawal from the study for any reason, (ii) The patient is lost to follow-up, (iii) The patient fails to comply with protocol requirements or study-related procedures, (iv) The Investigator determines that it is in the best interest of the patient to withdraw from the study protocol for reasons other than an adverse event (AE), (v) The study is terminated or temporarily suspended by the Sponsor or a regulatory, (vi) authority for any reason including, but not limited to, study drug-related unexpected life-threatening serious AEs (SAEs) detected during safety monitoring (e.g., torsade de pointes or other ventricular arrhythmias), and 2 or more of the same drug-related (possible or probable) treatment-emergent SAE.

Patients who wish to withdraw completely from this clinical study during the treatment period should be encouraged to undergo the EOT safety and efficacy assessments at the time of withdrawal. For a patient who agrees to complete the EOT visit, blood draws for PK will not be conducted. In addition, for women of child-bearing potential a pregnancy test should be conducted at this visit. Once a patient withdraws consent, no further data will be collected.

A patient is considered lost to follow-up if he or she fails to return to the study clinic for scheduled visits and is unable to be contacted by the study site. The site must attempt to contact the patient at least twice by phone or letter, and, if no response is obtained from the patient, a certified letter will be sent requesting that the patient contact the Investigator and asking that they return any unused IP and return to the site for a final evaluation. Documentation of these efforts to contact the patient must be recorded in the patient's study file.

Criteria for Stopping the Study. Possible reasons for stopping the study include, but are not limited to, the following: (i) Occurrence of an SAE assessed as probably related to IP and resulting in death, (ii) Occurrence of 2 or more SAEs of the same character that are determined to be clinically significant (CS) and assessed as probably related to IP, (iii) Occurrence of 2 or more AEs of acute pancreatitis, defined as the presence of abdominal pain, elevated pancreatic enzymes (amylase and lipase)≥5×ULN and determined to be CS, and an abdominal ultrasound with finding consistent with acute pancreatitis, (iv) Occurrence of 2 or more AEs of acute hepatitis meeting Hy's Law criteria, defined by at least 3-fold elevations of ALT or AST above the ULN, elevation of serum total bilirubin to >2×ULN without elevated serum alkaline phosphatase, and no other disease or condition can be found to explain the liver test abnormalities.

Identity of the Investigational Product. Investigational product refers to both SPR720 and placebo in this Investigational Product section. SPR720 is administered po as 250 mg capsules containing SPR720 as the active substance with no additional excipients. The capsules are white, opaque, size 0, hard gelatin capsules containing a white powder. Placebo doses is also administered po as white, opaque, size 0, hard gelatin capsules containing an approximately equivalent weight of microcrystalline cellulose (white powder). SPR720 drug product and placebo are manufactured by Patheon UK Limited (Abingdon, Oxfordshire, UK) under Good Manufacturing Practice conditions and packaged, labeled, and distributed by Almac Clinical Services. SPR720 and placebo are packaged in blister cards of 28 capsules each and then packaged into wallets given to the patient. Standard of care (SOC) will be obtained by the study sites or by prescription. Refer to the Product Package Inserts for detailed information regarding SOC treatments.

Test Product, Dose, and Mode of Administration. The SPR720 500 mg dose is administered as capsules, p.o., once daily for 28 days. The SPR720 1000 mg dose is administered as capsules, p.o., once daily for 28 days.

Recommended Reference Product, Dose, and Mode of Administration. Standard of Care (SOC) regimen is at the clinicians' discretion. Recommended standard of care is as follows: 2-drug or 3-drug SOC administered p.o. once daily for 28 days as follows: Clarithromycin 500-1000 mg, plus ethambutol HCl 15 mg/kg or Azithromycin 250-500 mg, plus ethambutol HCl 15 mg/kg. Optional rifampin 600 mg or rifabutin 300 mg p.o. once daily may be added to the SOC regimen for up to 28 days.

Criteria for Evaluation of Primary Pharmacokinetic Endpoints. Maximum plasma concentration (Cₘₐₓ), time to reach Cₘₐₓ (Tₘₐₓ), area under the concentration-time curve from zero to τ, where τ is the dosing interval (AUC0-τ), and accumulation ratio of SPR719 are assessed on Day 1 and Day 28.

Secondary Safety Endpoints. Secondary safety endpoints include reported adverse events (AEs), clinically meaningful changes in physical examination findings, concomitant medication usage, changes from baseline in laboratory tests, clinically significant (CS) out-of-normal range laboratory tests, shifts from baseline in selected laboratory tests using National Cancer Institute Common Terminology Criteria for Adverse Events (CTCAE) v5.0 shift categories, changes from baseline in vital sign measurements (body temperature, pulse, heart rate, respiratory rate, blood pressure), and 12-lead ECGs.

Exploratory Microbiological and Efficacy Endpoints. Microbiological and efficacy endpoints include (i) slope of the weekly sputum log₁₀ colony-forming unit (CFU)/mL from Day 1 through Day 28, from Day 1 through Day 14, and from Day 14 through Day 28 in the microbiological Intent-to-Treat (micro-ITT) Population, (ii) slope of the weekly sputum TTP from Day 1 through Day 28, from Day 1 through Day 14, and from Day 14 through Day 28 in the micro-ITT Population, (iii) slope of the weekly quantitative serum Nanodisk-MS assay from Day 1 through Day 28, from Day 1 through Day 14, and Day 14 through Day 28, in the micro-ITT Population, (iv) change from baseline (Day 14-Day 1; Day 28-Day 1) in the sputum log₁₀ CFU/mL in the micro-ITT Population, (v) change from baseline (Day 14-Day 1; Day 28-Day 1) in the sputum time-to-positivity (TTP) in the micro-ITT Population, (vi) change from baseline (Day 14-Day 1; Day 28-Day 1) in quantitative serum Nanodisk-MS assay in the micro-ITT Population, (vii) changes in susceptibility (≥4-fold increase in minimum inhibitory concentration for same pathogen identified at baseline) in SPR719 from Day 1 through Day 28, (viii) assessment of clinical response (defined as resolved, improved, unchanged, or worsened) at EOT in baseline signs and symptoms in the micro-ITT Population, and (ix) improvements in clinical symptoms and well-being using individual constructs and composite score from the multi-dimensional patient reported outcomes (PROs) from baseline to EOT in the micro-ITT Population. Note that for all exploratory microbiological and efficacy endpoints, the analysis and comparisons are between Treatment Arms 1, 2 and 3. All microbiological and efficacy data will be collected in Treatment Arm 4 (SOC), but this will be used to demonstrate assay sensitivity in a patient population treated for NTM-PD.

Schedule of Assessments. Assessments are conducted throughout the study on a regular schedule. Health assessments include height and weight checks, vital sign checks, 12-lead ECG, pregnancy test (females), urinalysis, blood for safety, blood samples for sparse PK, blood samples for biomarker, patient collected expectorated sputum, induced sputum, IP administration, and investigator assessment of clinical response.

Laboratory Data. Laboratory results (hematology, serum chemistry, and urinalysis) will be summarized by treatment arm for each time point, change from baseline at each postbaseline time point, shifts from baseline for selected laboratory tests (aspartate aminotransferase, alanine aminotransferase, gamma-glutamyl transferase, alkaline phosphatase, total bilirubin, amylase, lipase, blood urea nitrogen, serum creatinine, creatinine clearance, hemoglobin, hematocrit, neutrophils, lymphocytes, and erythrocytes) using CTCAE v5.0, and CS out-of-normal range laboratory. In addition, scatterplots of key laboratory values over time will are recorded.

Electrocardiographic Data. Baseline and change from baseline in ECG parameters (heart rate, cardiac rhythm, PR interval, RR interval, QRS interval, QT interval, and QTc interval) will be summarized at each postbaseline time point. At each time point, the number of patients with QTc according to Fridericia's formula (QTcF) values of >450 ms, >480 ms, and >500 ms will be presented. At each postbaseline time point, the number of patients with change from baseline values in QTcF of >30 ms and >60 ms is recorded.

Statistical Methods. Presentation of PK and safety data will be produced for the PK Population and Safety Population, respectively. A PK Population model using data from Example 1 of single-ascending/multiple-ascending doses in normal healthy volunteers is used to estimate PK parameters for patients in this example. Dates and times of blood samples as well as dates and times of study drug administration will be recorded. Plasma concentration-time data from intensive PK sampling patients will be subjected to noncompartmental PK analysis. Day 1 and Day 28 PK parameters including, but not limited to, Cₘₐₓ, Tₘₐₓ, area under the concentration-time curve from zero to last concentration (AUCₗₐₛₜ), AUC_{0-τ}, accumulation ratio, and plasma concentration at the end of a dosing interval at steady state (taken directly before next administration) (C_{trough}) will be determined for SPR719. Concentration data from sparse sampling will be compiled for summary statistics of C_{trough} across various days. Concentration data from SPR720 recipients are used for a PK Population model, for PK analysis, and for estimation of individual PK profiles in patients with sparse sampling. Patient disposition, demographics and baseline characteristics, IP exposure, and prior and concomitant medications will be summarized.

The main exploratory microbiological analyses are the comparison of the change in mean CFUs/mL in weekly quantitative cultures from respiratory samples (pooled expectorated sputum, induced sputum, and mean sputum [expectorated and induced]) in micro-ITT Population. The CFU data is log transformed before analysis. In addition, a comparison of the change in mean TTP in liquid media (MGIT) from respiratory samples (pooled expectorated sputum, induced sputum, and mean sputum [expectorated and induced]) and change in mean quantitative Nanodisk-MS assay from serum in the micro-ITT Population is undertaken. Linear and bi-linear models or longitudinal analysis are considered to describe the profile of log₁₀ CFU counts over time.

Extent of Exposure. The extent of exposure to study is summarized by the number of doses taken, number of missed doses, and compliance with IP administration. Investigational product noncompliance for a patient is defined as taking less than 80% or more than 120% of IP during the treatment period (Day 1 through EOT).

1-12. (canceled)

13. A method of treating an infection in an adult human patient, comprising orally administering to the patient 500 mg SPR720 once daily for at least 14 days or 1000 mg SPR720 once daily for at least 14 days, where the infection is a tuberculous infection of a nontuberculous mycobacteria pulmonary disease (NTM-PD),

14. The method of claim 13, wherein the 500 mg SPR720 is orally administered once daily for at least 28 days.

15. The method of claim 13, wherein the 1000 mg SPR720 is orally administered once daily for at least 28 days.

16. The method of claim 13 wherein, the infection is a tuberculosis infection.

17. The method of claim 13, wherein the infection is a Mycobacterium avium complex (MAC) infection.

18. The method of claim 17, wherein the MAC infection includes infection with one or both of M. abscessus and M. kansasii.

19. The method of claim 13, wherein the patient has an underlying condition selected from immunodeficiency, chronic obstructive pulmonary disease (COPD), cystic fibrosis, cavitary or fibo-cavitary lung disease, current or previous treatment with an antitumor necrosis factor agent, neutropenia, a hematologic malignancy, a history of hematopoietic stem cell transplant, a history of solid organ transplant, current treatment with immunosuppressive therapy, current treatment with cancer chemotherapy, current treatment with monoclonal antibodies for autoimmune disease, previous or current C. difficile infection, hepatic impairment as evidenced by alanine aminotransferase or aspartate aminotransferase>2× upper limit of normal (ULN) or total bilirubin>1.5×ULN, clinical signs of cirrhosis or end-stage hepatic disease (e.g., ascites, hepatic encephalopathy), renal impairment as evidenced by a creatine clearance of <50 mL/min., and end-stage renal disease requiring hemodialysis or peritoneal dialysis.

20. The method of claim 13, wherein treatment with SPR720 is continued until the patient experiences improvement in at least one of the following symptoms: cough, hemoptysis, fatigue, chest pain, poor appetite, fever, night sweats, and weight loss.

21. The method of claim 13, wherein the patient is also administered an additional agent selected from amikacin, azithromycin, rifampin, rifabutin, ethambutol HCl, clarithromycin, streptomycin, amikacin, or an inhaled steroid.

22. The method of claim 21, wherein the daily dose of the additional agent is 500 mg to 1000 mg clarithromycin, 10 mg/kg to 20 mg/kg ethambutol hydrochloride, 250 mg to 500 mg, 100 mg to 1000 m rifampin, or 100 mg to 500 mg rifabutin.

23. The method of claim 13, wherein the NTM-PD is nodular bronchiectatic NTM-PD.

24. The method of claim 13, wherein the NTM-PD is fibro-cavitary NTM-PD.

25. A method of treating a nontuberculous mycobacterial infection in a patient comprising:
(i) determining that the nontuberculous mycobacterial infection is resistant to at least one of amikacin, clarithromycin, and fluoroquinolone antibiotics; and
(ii) orally administering to the patient 500 mg SPR720 once daily for at least 14 days or 1000 mg SPR720 once daily for at least 14 days.

26. A method of treating an NTM infection in a patient comprising administering
(i) a once daily oral dosage of SPR720 or a pharmaceutically acceptable salt thereof, the oral dosage of SPR720 comprising 500 mg to 1000 mg SPR720;
(ii) a once daily oral dosage of clarithromycin or a pharmaceutically acceptable salt thereof, the oral dosage of clarithromycin comprising 500 mg to 1000 g clarithromycin; and
(iii) a once daily oral dosage of ethambutol HCl comprising about 15 mg/kg ethambutol.
